(19) United States
(12) Patent Application Publication
(10) Pub. No.: US 2011/0092976 A1
(43) Pub. Date: Apr. 21, 2011

(54) Kit of alignable tools for preparing an intervertebral site for implanting a prosthetic intervertebral disc

(76) Inventors: Thomas Rawles, Plano, TX (US); Steve Moscaret, San Mateo, CA (US); Darin C. Gittings, Sunnyvale, CA (US)

(21) Appl. No.: 12/927,207

(22) Filed: Nov. 8, 2010

Related U.S. Application Data
(63) Continuation of application No. 11/970,220, filed on Jan. 7, 2008.

(51) Int. Cl. A61B 17/16; A61B 17/56

(52) U.S. Cl. 606/83; 606/87

(57) ABSTRACT

We describe various alignable tools, in particular, trials and chisels, that may be used in preparing an intervertebral site in a spine, the site being suitable for placement of a prosthetic intervertebral disc as a replacement for a natural disc in that spine. We also describe methods of using those tools.

### RELATED APPLICATIONS

This a continuation of U.S. Ser. No. 11/970,220, filed Jan. 7, 2008, the entirety of which is incorporated by reference for all reasons.

### FIELD

We describe various alignable tools, in particular, trials and chisels, that may be used in preparing an intervertebral site in a spine, the site being suitable for placement of a prosthetic intervertebral disc as a replacement for a natural disc in that spine. We also describe methods of using those tools.

### BACKGROUND

The intervertebral or spinal disc is an anatomically and functionally complex joint. The intervertebral disc is made up of three component structures: (1) the nucleus pulposus; (2) the annulus fibrosus; and (3) the vertebral end plates. The biomedical composition and anatomical arrangements within these component structures are related to the biomechanical function of the disc.

The spinal disc may be displaced or damaged due to trauma or disease. As a result of such displacement or damage, the nucleus pulposus may herniate and protrude into the vertebral canal or intervertebral foramen. Such a deformation is known as a herniated or “slipped” disc. This protrusion may press upon one or more of the spinal nerves exiting the vertebral canal through a partially obstructed foramen, thereby causing pain or paralysis in the area of its distribution. Similarly, spinal discs may degenerate with age or excessive use resulting in a decrease in disc height. One specific result of such disc height decline is the narrowing of the foramenal space, often causing pressure on the emanating nerve and causing pain and paralysis in the area of the nerve's influence. Pressure on the nerve and disc herniation often happen together.

Artificial intervertebral discs are used to replace damaged or diseased natural intervertebral discs. Various types of artificial intervertebral discs have been developed with the goal of restoring the normal kinematics and load-sharing properties of the natural intervertebral discs. Two such types are ball-and-socket joint type discs and the elastomer type discs.

We have described prosthetic intervertebral discs in U.S. Pat. No. 7,153,325, entitled “Prosthetic Intervertebral Disc and Methods for Using Same,” issued on Dec. 26, 2006, assigned to Spinal Kinetics Inc., and in U.S. patent application Ser. No. 10/903,276, entitled “Prosthetic Intervertebral Disc and Methods for Using Same,” filed Jul. 30, 2004, published as 2005/0228500 on Oct. 13, 2005; Ser. No. 11/218,707, entitled “Prosthetic Intervertebral discs,” filed Sep. 1, 2005, published as 2007/0050032 on Mar. 1, 2007; Ser. No. 11/281,205, entitled “Prosthetic Intervertebral Discs,” filed Nov. 15, 2005, published as 2007/0050033 on Mar. 1, 2007; Ser. No. 11/527,804, filed on Sep. 26, 2006, entitled “Prosthetic Intervertebral Discs having Substantially Rigid End Plates and Fibers Between Those End Plates,” published as 2007/0168033 on Jul. 19, 2007; and Ser. No. 11/528,100, filed Sep. 27, 2006, entitled “Prosthetic Intervertebral Discs having Stiff End Plates and a Compressible Polymeric Core,” published as 2007/0168034 on Jul. 19, 2007, the entirety of which are incorporated by reference.

### SUMMARY

Below, we describe alignable tools and tool systems, particularly chisels and trials, that may be used variously for determining the proper size and position of a prosthetic disc to be implanted and for preparing that site, e.g., by chiseling, for the placement of the prosthetic intervertebral discs. Generally, the trial and the chisel are used free-hand, in the sense that for many variations of the described tools, the tools are not used simultaneously by the surgeon and are positioned by hand rather than through the use of jigs and other tools. These tools use a visualization device, e.g., a fluoroscope, in conjunction with the spine itself to align the tools and to assure proper sizing and placement of the prosthetic disc. The tools are alignable via, for instance, the presence of alignment passageways in the tools allowing a sequence of the tools to be viewed in situ with a fluoroscope and positioned or repositioned as necessary to align the latter tools in the former tools' position.

In particular, the tools may comprise trials and chisels. Trials are individual tools used to determine the overall size of the intervertebral space and to provide guidance to the surgeon in selecting the size of the prosthetic intervertebral disc to be implanted. Since the intervertebral space usually exhibits lordosis and hence is generally wedge-shaped, various sizes of trial, perhaps with the same included angle between the trial faces, are also used to select a proper anterior-posterior (“AP”) placement of the prosthetic intervertebral disc to be implanted. Qualitatively, a thicker trial resides in a more anterior end of the intervertebral space; conversely, a thinner trial resides more towards the posterior of the space.

A progression of different sized trials is inserted into the disc space to determine the appropriate size, including height, lordotic angle, anterior-posterior and lateral dimensions, and the position of the prosthetic disc to be implanted. The different sized trials may have shapes corresponding to different lordotic angles and sizes.

Specifically, one procedure for using the set of trials is thus: the centerline of the vertebral bodies may be marked on those vertebral bodies. A centerline mark on the trial may be then aligned with the centerline of the vertebral bodies to center those trial. That first trial is inserted into an intervertebral space after the native disc has been removed. The placement of the trial head in the vertebral space is assessed, by reviewing the degree of correspondence between the trial's height and included angle (i.e., between the trial faces) and the observed height and angle between the vertebral surfaces and by considering the AP placement of the trial head. If the angle or height is not that desired, a trial with a different angle or height is chosen and the resulting placement is reviewed. If the trial sits too far towards the anterior, a smaller (or thinner) trial is selected for review. The surgeon confirms choice of the appropriate trial. This trial determines the size of the prosthetic disc. The surgeon also confirms that the trial is situated appropriately in the disc space, e.g., generally centered in the intervertebral space (if the replacement is a one-piece prosthetic disc), and aligned to the sagittal plane of the patient.

The position of our alignable trial is then determined by placing a fluoroscope is a position such that is aligned with the trial's alignment passageway. Often that alignment passageway is side-to-side in the trial head, but it need not be. The trial is then removed.

The chisel head is inserted into the space typically with a hammer to drive the chisel into the space and to cut grooves into the opposing faces on the vertebral bodies. If the chisel includes a stop, the chisel is driven until the stop on the chisel head engages a vertebral surface or surfaces. This indicates that the chisel head has progressed to a specific chosen depth and, therefore, produced the chosen length of groove in the vertebral body. These linear grooves in the face of the vertebrae are intended variously for proper alignment of the prosthetic disc in the intervertebral space during deployment, for proper depth (or AP placement) of the prosthetic disc in the opening, and for fixation of the disc by sliding one or more fixation elements, such as fins, keels, anchors, pins, barbs, screws, etc., situated on the disc face into the chiseled linear grooves. The groove length is often chosen to match a corresponding measurement on the prosthetic disc attachment component.

Another chisel head may be inserted into the disc space to cut additional grooves into an adjoining vertebral body. That other chisel head may also include a stop for controlling the length of the groove.

As noted above, such placement may, for chisels having exterior cutting surfaces, involve pounding the chisel into the intervertebral space until the corresponding alignment passageway is aligned with the view provided by the fluoroscope. If a chisel includes cutting surfaces that are extended outwardly from the face of the chisel into vertebral bone after placement in the intervertebral space, the initial placement of the chisel obviously requires less force.

After the chisel is removed, the prosthetic disc is placed in the intervertebral space.

The tool systems may include one or more chisels or one or more trials. Typically, a tool system will include at least one trial and at least one chisel corresponding in shape or size to the trial. A tool system may include collections of multiple trials and of multiple chisels. Other tool systems may include a number of trials, often of different or incremental sizes, or a number of chisels, also of different or incremental sizes.

The described trial or trials may include one or more fluoroscopically visible passageways through the trial in a position matched by one or more fluoroscopically visible passageways through a corresponding chisel or chisels. The passageways in each of the trial and corresponding chisel may, for instance, be circular and pass from side-to-side allowing a surgeon user to match the position of a chisel to that of the appropriate trial. The passageways may be at least partially filled with a fluorolucent material.

Although the trials and chisels will generally be radio-opaque and typically metallic, with open or radiolucent alignment passageways, that need not be the case. The body may be at least partially radiolucent but have radio-opaque features, such as alignment pins, for indicating AP depth and lateral alignment of the trial between the vertebral bodies.

One or more chisels may be used to cut grooves in the vertebral bodies, which grooves are, in turn, used for aligning and affixing the disc to the vertebral bodies. The chisels may include external surfaces or cutters for creating the grooves. The number, orientation, and shape of the cutters may match the attachment or fixation component of the prosthetic disc to be implanted. The size of the cutters may be a percentage of the size—in width, height, or both—of the attachment component on the disc, ranging anywhere from 50-125%, and perhaps approximately 80% to 100%, often about 100%. The chisels may include “stops” that contact, for instance, one or more surfaces on the vertebrae when the chisel is inserted into the intervertebral space at a predetermined depth. Such a stop may be used to control the length of a groove cut by the chisel in the disc space.

The chisels are typically wholly or partially radio-opaque with passageways corresponding to those in a matching trial so that a user may visualize those chisels under fluoroscopy to ensure correct positioning, both laterally and anterior-posteriorly and matching the positioning of that trial.

We describe a chisel assembly having extendable cutting members. These allow introduction of the chisel into the intervertebral space and proper alignment of the chisel to take place with great ease. The cutting members may be extended by a cam or the like situated in the chisel handle. Removing the chisel may take place with the aid of a slide hammer or the like.

The tool systems may optionally include a distractor for moving or “distracting” two adjacent vertebral bodies in a spine thereby providing access to an intervertebral space for implanting the prosthetic disc. One variation of the distractor is made up of an upper jaw, a lower jaw, a mechanism for opening the upper and lower jaws, and a mechanism for maintaining the opening between those upper and lower jaws. The upper and lower jaws contact the two adjacent vertebrae and are used to press them apart.

The tool systems may also include an inserter tool for placing the prosthetic disc into the disc space between the vertebral bodies. The inserter may include engagement features configured to cooperate with mating features on the prosthetic disc's end plates. Those inserter engagement features and prosthetic disc mating features may be arranged so that, when the disc is inserted into its selected disc space, the prosthetic disc itself is in a compressed, hyper-lordotic state that eases that final implantation passage.

Other and additional devices, apparatus, structures, and methods are described by reference to the drawings and detailed description below.

### BRIEF DESCRIPTION OF THE DRAWINGS

FIG. 1 shows several views the our described trial. In particular, the figure shows top view width a removable handle attached, a distal and view of the trial ahead, a proximal and view of the trial head, and bottom view of the trial head, a cross section (longitudinal) of the trial head, and a perspective view of the trial head.

FIG. 2 shows several views of one variation of our described chisel. In particular, the figure shows a top view with a fixed the handle, a distal end a view of the chisel head, a proximal, cross-sectional, end view of the chisel head, a top view of the chisel and, a side view of the chisel head, and a perspective view of the chisel and.

FIG. 3 depicts a procedure for utilizing the described trials and chisels in preparing an intervertebral site for placement of a prosthetic replacement disc.

FIGS. 4A-4E show a number of suitable radiolucent pathways or passageways for our described tools.

FIGS. 5A and 5B show a chisel having extendable cutting surfaces.

FIGS. 6A and 6B show side, cross-sectional views of one variation of our mechanism for extending the cutting surfaces of the chisel of FIGS. 5A and 5B. FIG. 6A shows the chisel of FIGS. 5A and 5B before the cutting surfaces are extended and FIG. 6B shows the device after the cutting surfaces are extended.

FIGS. 7A₁ and 7B₁ also show side, cross-sectional views of another variation of a mechanism for extending the cutting surfaces of the chisel of FIGS. 5A and 5B. FIGS. 7A₂ and 7B₂ show, respectively, end views of the mechanism shown in FIGS. 7A₁ and 7B₁ before and after the cutting surfaces are extended.

FIG. 8 depicts a procedure for using the devices shown in FIGS. 5A to 7B₂.

FIGS. 9A and 9B show, respectively, fluoroscopic side views of an improper placement of our described chisel with respect to a spine and of a proper placement of the chisel.

FIG. 10 shows an anterior, fluoroscopic view of our trial situated in a spine. The trial has had its handle removed.

FIG. 11 shows an anterior, fluoroscopic view of our trial situated in a spine. The depiction also shows the location of several other surgical devices, e.g., spreaders.

### DETAILED DESCRIPTION

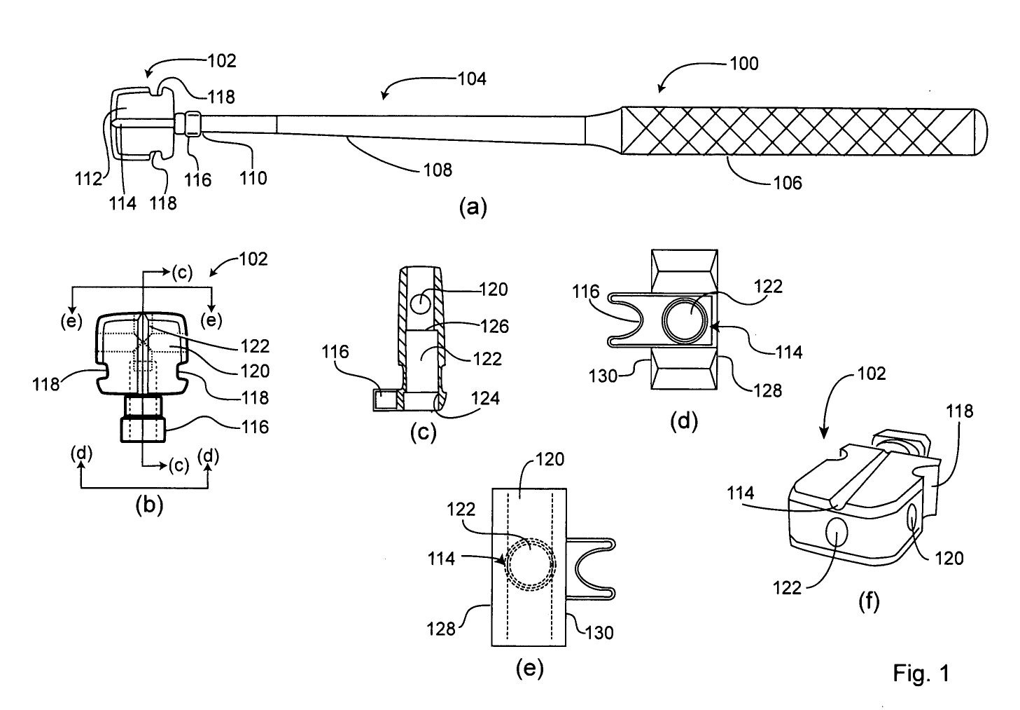

FIG. 1 shows a number of views of one variation of our trial (100). View (a) shows a top view of our trial (100) and further depicts the trial head (102) and a removable handle (104). In this variation, handle (104) is shown to have a grasping surface (106), made so by knurling or grooving, a narrowed shaft (108), and a joining region (110). The joining region (110) is configured to be removable from the trial head (102) perhaps by screw threads or a bayonet mount or other suitable detachable joints.

Trial head (102) is shown in view (a) to be attached to handle (104). This is the assembled form of this variation of our trial (104). This view of the trial head (102) shows a surface (112) that contacts one of the prepared vertebral surfaces forming the upper and lower bounds of the intervertebral space. The groove (114) in surface (112) is provided to allow the surgeon visually to center trial head (102) by lining up groove (114) with a center mark on the chosen vertebrae previously made by the surgeon. This view (a) also shows marking guide (116) that may be used by the surgeon in making a mark on the vertebra. Notches (118) may be used as grasping sites for an implement, e.g., grasping pliers or the like, that may be used to remove the trial head (102) from the human body in the event that the handle (104) cannot be reattached for removal from the intervertebral space or the user wishes to remove the trial head (102) for other reasons.

View (b) provides a top view of trial head (102) with the handle (104) removed. Groove (114) and marking guide (116) may also be seen in this view as may grasping notches (118). The outlines of the lateral opening or sighting passageway (120) and the longitudinal sighting passageway (122) are also shown in this depiction. The lateral passageway (120) and longitudinal sighting passageway (122) are used in the alignment procedure described below.

In this view, the passageways are shown to include a substantially right angle between them. They are also shown to be substantially round. As is noted elsewhere, the passageways need not be at a right angle to each other. The various sighting bores need not be round. First, the lateral passageway (120) may situated at an angle selected to allow, for instance, a more vertical placement of the fluoroscope. If the longitudinal sighting passageway (122) is positioned as shown in FIG. 1, view (d), the transversely located sighting passageway (120) may be rotated to an angle between 15° to 20° and 90°. However, the closer that that angle is to 90°, the more accurate the sighting step is believed to be.

Further, the longitudinal sighting passageway (122) is shown to have a pair of steps (124, 126) separating regions having differing diameters. These steps are for placement of the handle (104 in FIG. 1, view (a)) and serve as stops, preventing the passage of the handle through the trial head (102) during placement.

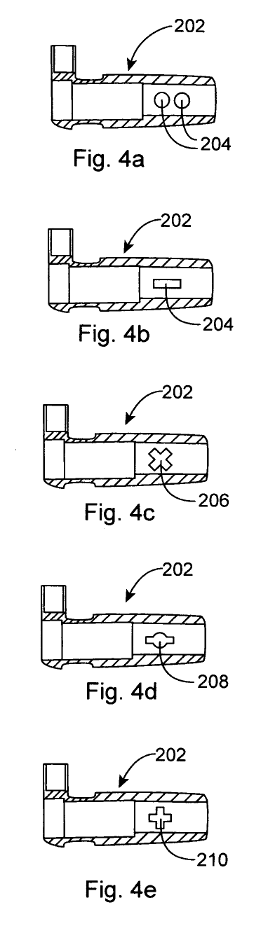

As will be discussed with regard to FIG. 4 below, the shape of the transverse bore need not be round, but may be of another effective shape, allowing perhaps even greater accuracy of sighting and of disc placement.

In any case, returning to FIG. 1, view (d) shows a front end view (or, view from the handle) and view (e) shows a back end view (or distal end view in the sense of being distal from the user) of the trial (102). The longitudinal sighting passageway (122), groove (114), and marking guide (116) may also be readily observed.

Finally, view (f) of FIG. 1 shows a perspective view of the trial (102) with the longitudinal sighting passageway (122), lateral passageway (120), groove (114), and marking guide (116).

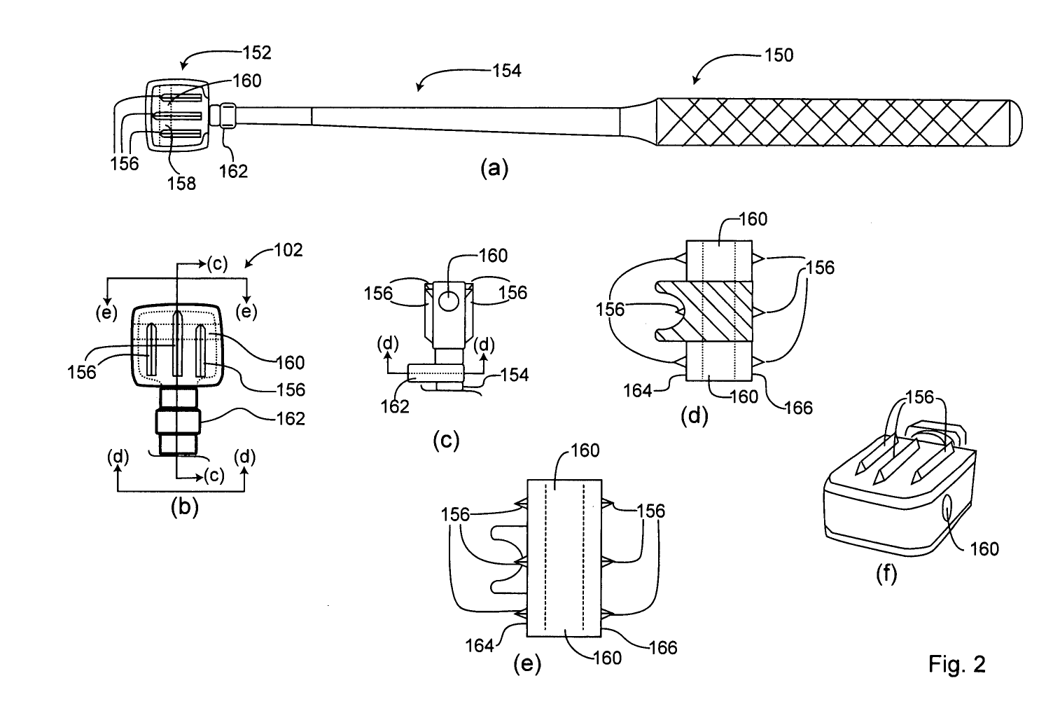

FIG. 2 shows a number of views of our chisel (150) and its isolated chisel head (152). The chisel (150) may either be made up of a chisel head (152) that is separable from the handle (154) or the chisel head (152) may be integral with (or otherwise inseparable from) the handle (154).

View (a) of FIG. 2 shows a top view of chisel (150) with its component handle (154) and chisel head (152). This chisel is used, in particular, to form grooves in the facing surfaces of two adjacent vertebrae. Those grooves accept fixation components (e.g., fins or keels) that may be placed on or integrated into prosthetic intervertebral discs as shown in our U.S. Pat. No. 7,153,325, entitled “Prosthetic Intervertebral Disc and Methods for Using Same,” issued on Dec. 26, 2006, assigned to Spinal Kinetics Inc., and in U.S. patent application Ser. No. 10/903,276, entitled “Prosthetic Intervertebral Disc and Methods for Using Same,” filed Jul. 30, 2004, published as 2005/0228500 on Oct. 13, 2005; Ser. No. 11/218,707, entitled “Prosthetic Intervertebral discs,” filed Sep. 1, 2005, published as 2007/0050032 on Mar. 1, 2007; Ser. No. 11/281,205, entitled “Prosthetic Intervertebral Discs.” As a disc with these fixation components is slipped into an intervertebral space prepared with this chisel (150), the fins or keels follow the vertebral grooves or furrows formed by this chisel and eventually fix the prosthetic disc in place by a combination of mechanical forces and bone in growth.

The cutting surfaces or structures (156) on chisel head (152) are shown to be generally triangular in cross-section but may be of any shape that cooperates with the fixation components found on the allied prosthetic disc. Similarly, chisel head (152) is shown to have three cutting surfaces (156) on each of its faces. Although this is a suitable number, the number and position of cutting surfaces (156) on may be chosen to match or to complement the number and position of fixation components found on the prosthetic disc. The lateral sighting passageway (160) is seen, in shadow, in chisel head (152).

View (b) of FIG. 2 shows a top view and view (c) shows a side view of the chisel head (152), three cutting surfaces (156), centering alignment component (162), and lateral alignment bore or passageway (160) in shadow. The distal end of handle (154) may also be seen.

View (d) of FIG. 2 shows a cross-sectional view of the chisel head (152) taken through the centering alignment component (162) viewing towards the distal end of the chisel (150). The three cutting surfaces (156), centering alignment component (162), and lateral alignment bore or passageway (160) in shadow may also be seen.

View (e) shows a distal end view and view (f) provides a perspective view of the chisel head (152) also showing three cutting surfaces (156), centering alignment component (162), and lateral alignment bore or passageway (160) in shadow.

The faces (128, 130 in FIG. 1, views (d) and (e)) of trial head (102) may be substantially parallel to each other, particularly when the trial is to be used in the cervical region of the spine. The trial faces may have up to about 5° to 10° of lordosis. In such instances, the angles between faces (164, 166 in FIG. 2, views (d) and (e)) of chisel head (152) typically would mirror of those in the corresponding trial, although a small angle of up to about 5° between the chisel faces (164, 166) may be added to provide for, e.g., chisel control. When these tools are to be used in the lumbar region of the spine, the angles between the trial head faces and between the chisel head faces each often range between about 2° and about 15°, or between about 3° and about 13°, to provide a determined amount of lordosis to the spine when a similarly angled prosthetic disc is implanted. Also, the respective thicknesses of the trial head and of the chisel head are chosen to reflect the thickness of the later implanted prosthetic disc, e.g., between about 10 mm. and 14 mm.

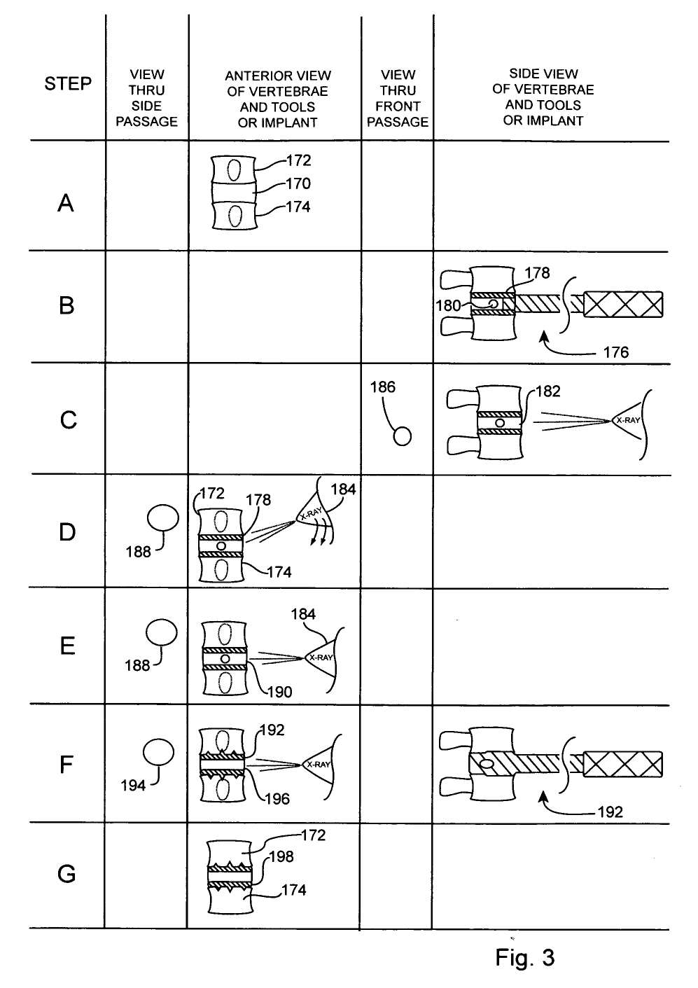

FIG. 3 shows a typical procedure for using the trials and chisel we have described here.

Step A provides an anterior view of the site for implanting a prosthetic disc. The intervertebral space (170) has been prepared by removal of the natural disc from between upper vertebra (172) and lower vertebra (174).

Step B shows a side view of our trial (176) with the trial head (178) inserted into the intervertebral space (170). The size of the trial (176) has, via a combination of pre-selection, experience, and trial-and-error, been selected. Step C shows a side view of the trial head (178) residing in the intervertebral space (170). The handle of the trial (176) has been removed to allow sighting of the fluoroscope (184) through the anterior-posterior sighting port (182). The shape of the view (186) may also be seen.

Step D shows an anterior view of the trial head (178) inserted into the intervertebral space (170) between upper vertebra (172) and lower vertebra (174). The fluoroscope (184) is out of position as evidenced by the oval shape of view (188).

Step E shows the same view as does Step D, but the fluoroscope (184) has been relocated into a proper position aligned with the side-to-side alignment bore (190) as evidenced by the roundness of the sight (188) through that bore.

Step F shows anterior and side views of the chisel (192) situated in the intervertebral space (170). As evidenced by the round shape of the view (194) through the side-to-side bore (196), the chisel (192) approached and now resides in the intervertebral space (170) in the same position and alignment as did the trial (174) in the prior steps.

Step G shows an anterior view of a prosthetic intervertebral disc (198) situated in the intervertebral space (170) between upper vertebra (172) and lower vertebra (174) after the chisel (174) has been removed.

FIGS. 4A-4E show examples of various passageway shapes suitable for our trial and chisel.

FIG. 4A shows a cross-section side-view of an exempletive tool (202) having a plurality of centering alignment ports or openings (204), e.g., two such passageways, passing from side-to-side in the tool (202). The depicted shapes of multiple ports are shown to be round although they need not be. The multiple ports need not be of the same type or size. Round ports, whether single or multiple, are particularly suitable since the native eye is easily to discriminate between round and oval.

FIG. 4B shows a cross-section side-view of an exempletive tool (202) having a rectangular alignment port or opening (206) passing from side-to-side in the tool (202). Such a shape as is shown in the drawing, with the narrower dimension of the rectangle extending vertically, provides a differential or distinctiveness in the positional fineness with which the fluoroscope (and hence the implanted disc) may be placed in considering the up and down placement along the side, in comparison to the wider horizontal opening which is more tolerant of misalignment in the anterior-posterior direction.

FIG. 4C shows a cross-section side-view of an exempletive tool (202) having a cross-shaped alignment port or opening (208) passing from side-to-side in the tool (202). The cross components are diagonal to the placement position in the spine.

FIG. 4D shows a cross-section side-view of an exempletive tool (202) having a combination alignment port or opening (210) passing from side-to-side in the tool (202). The opening is a combination in that it includes a round portion that is visually round when viewed along the axis of the round portion and is visually oval when viewed through the end of the port but off of that section's axis. The open rectangular portion of the port (210) has a very small vertical component and hence is invisible when the port is viewed with but a minor misalignment.

FIG. 4E shows a cross-section side-view of an exempletive tool (202) having a cross-shaped alignment port or opening (212) passing from side-to-side in the tool (202). The cross components are vertical and horizontal to the placement position in the spine.

The chisel tools shown in the various views in FIG. 2 discussed above depict chisel surfaces or cutting surfaces (156) that are integral with or otherwise fixed upon the chisel head (152). Those chisel cutting surfaces (156) are immobile with respect to the chisel face (152). This style of chisel cuts grooves in the vertebral surfaces when entering the intervertebral space.

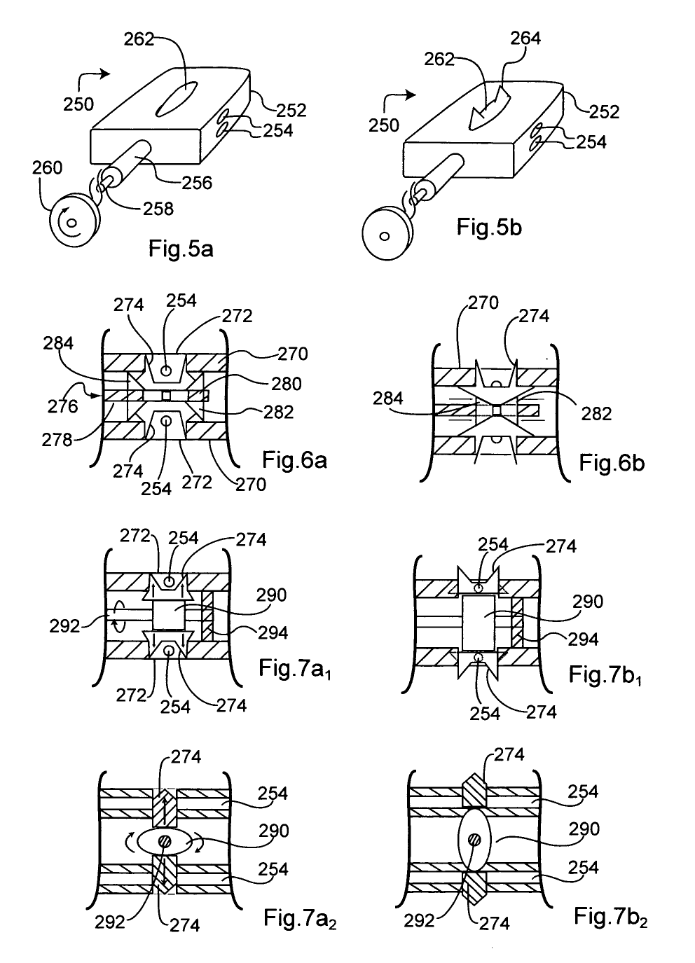

Another variation of our chisel utilizes extendable cutting surfaces that cut grooves when being removed from the intervertebral space. The chisels may be used to cut patterns in the vertebral surface that are complementary to fixed or deployable anchoring features in the implant, thus lowering the risk of implant migration. This variation of our tool may also be used as a trial when the extendable cutting surfaces are retracted. FIGS. 5A and 5B provide a generic depiction of our chisel, respectively, with the movable cutting surfaces retracted and with the movable cutting surfaces extended. FIGS. 6A and 6B show the structure and the manner of extending the movable cutting surfaces of one form of the chisel shown in FIGS. 5A and 5B. FIGS. 7A₂ to 7B₂ show the structure and the manner of extending the movable cutting surfaces of another form of the chisel shown in FIGS. 5A and 5B.

FIG. 5A shows a chisel (250) having a chisel head (252) and a pair of alignment ports (254) situated in the chisel head (252). This variation of the chisel (250) includes a handle (shown in partial section) with an outer shaft (256) and an outer shaft (258) attached to an activating knob (260). The outer shaft (256) is depicted to be immobile with respect to the chisel head (252); the inner shaft (258) is turned by knob (260) to extend the chisel cutting surfaces (264 in FIG. 5B) through opening (262) in the face of chisel head (252). Typically, the face of chisel head (252) that is not seen in the views provided in FIGS. 5A and 5B would also include an opening such as opening (262) for the extension of additional chisel cutting surfaces (264). Further, although only one set of extendable chisel cutting surfaces (264) is shown in the Figures, multiple rows of such chisel cutting surfaces (264), e.g., two chisel cutting surface rows possibly equidistant from the axis of the tool or three chisel cutting surface rows including one at the center line of the tool and two chisel cutting surface rows equidistant from the axis of the tool, are also suitable.

FIG. 5B shows the device shown in FIG. 5A but with the chisel cutting surfaces (264) extended through opening (262). With the chisel cutting surfaces (264) extended, the chisel (250) is ready to be removed from an intervertebral space and cut grooves as it is withdrawn.

FIGS. 6A and 6B show a partial cross-sectional view of the operation of the extendable chisel cutting surfaces. Specifically, FIG. 6A shows opposing chisel head faces (270) with an opening (272) in each face (270) for extension of the chisel cutting surfaces (274). The centering alignment ports (254) are also visible. In this mechanism, a central shaft (276) having sections (278, 280) with opposite-handed threads and cooperating, threaded conical members (282, 284) that, when the central shaft is twisted, the two interiorly threaded conical members (282, 284) are moved towards each other and squeeze the chisel cutting surfaces (274) up through openings (272). The chisel cutting surfaces (274) may have cooperating ramp surfaces adjacent to the exterior surfaces of the two interiorly threaded conical members (282, 284) to assist in extending the chisel cutting surfaces (274). FIG. 6B shows the two interiorly threaded conical members (282, 284) have approached each other and pushed the chisel cutting surfaces (274) through opening (272) in each face (270) exterior of the chisel head.

FIGS. 7A₁ and 7B₂ show another variation of our chisel tool having extendable cutting surfaces. In this variation a cam (290) is used to extend the chisel cutting surfaces (274) up through openings (272).

FIG. 7A₁ shows a partial, cross-sectional, side view of the chisel tool showing chisel cutting surfaces (274) and openings (272). A cam (290) having two lobes is rotated on a shaft (292) with a support (294) to push the chisel cutting surfaces (274) out of the chisel body through the opposed openings (272). FIG. 7A₂ shows a partial, cross-sectional, end view of the chisel shown in FIG. 7A₁ and, in particular, shows the double-lobed cam (290) and its direction of rotation about the shaft (292) to extend the chisel cutting surfaces (274).

FIG. 7B₁ shows a partial, cross-sectional, side view of the chisel tool found in FIG. 7A₁ with the cam (290) turned to extend the chisel cutting surfaces (274) through openings (272). FIG. 7B₂ shows a partial, cross-sectional, end view of the chisel tool found in FIG. 7A₂ with the cam (290) turned to extend the chisel cutting surfaces (274) through openings (272).

FIGS. 6A, 6B, 7A₁, 7A₂, 7B₁, and 7B₂ each show a pair of alignment ports (254) in the chisel. Although a pair of ports is shown, the alignment feature of the chisel is also operable with one such port or with more than one such port. Additionally, the chisel variations with extendable chisel cutting surfaces (274) shown in those Figures have separate utility without alignment ports.

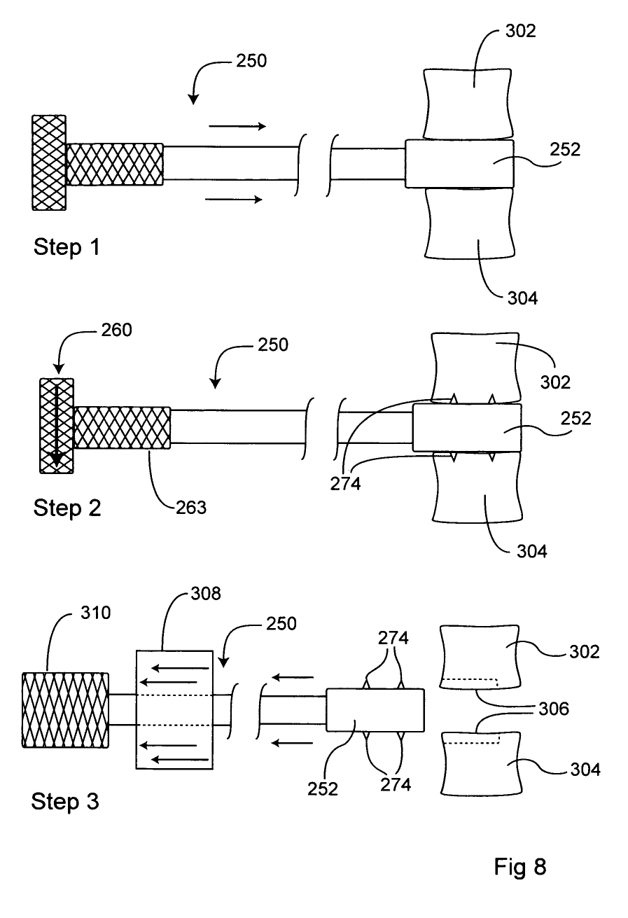

FIG. 8 schematically depicts a method of using the chisel tools with extendable chisel cutting surfaces as shown in FIGS. 5A, 5B, 6A, 6B, 7A₁, 7A₂, 7B₁, and 7B₂. Step 1 of FIG. 8 shows a side view of the chisel (250) shown in FIG. 5A. The chisel head (252) has been inserted into the intervertebral space situated between an upper vertebra (302) and a lower vertebra (304). That intervertebral space typically has been sized and measured with a trial to permit introduction of a chisel (250) of appropriate dimensions.

Step 2 shows the extension of the cutting surfaces. The knob (260), as also shown in FIGS. 5A and 5B includes an outer knob (261) that, when turned with respect to the inner knob (263), turns an inner shaft ((278) in FIG. 6A, (292) in FIGS. 7A₁, 7A₂, 7B₁, and 7B₂, but not shown in FIG. 8) to extend the cutting surfaces (274) into the vertebral surfaces facing the chisel head (252).

Step 3 shows removal of the chisel (250) and the chisel head (252) with cutting surfaces (274) extended. Removal of the chisel (250) cuts grooves (306) into upper vertebra (302) and lower vertebra (304). In the depicted variation, knob (260) has been replaced by a slide hammer assembly made up of slide weight (308) and stop (310). Slide weight (308) has substantial mass and as it is impelled against stop (310), pulls the chisel from the intervertebral space. That intervertebral space is now ready for placement of a disc implant having placement extensions such as keels, fins, and spikes.

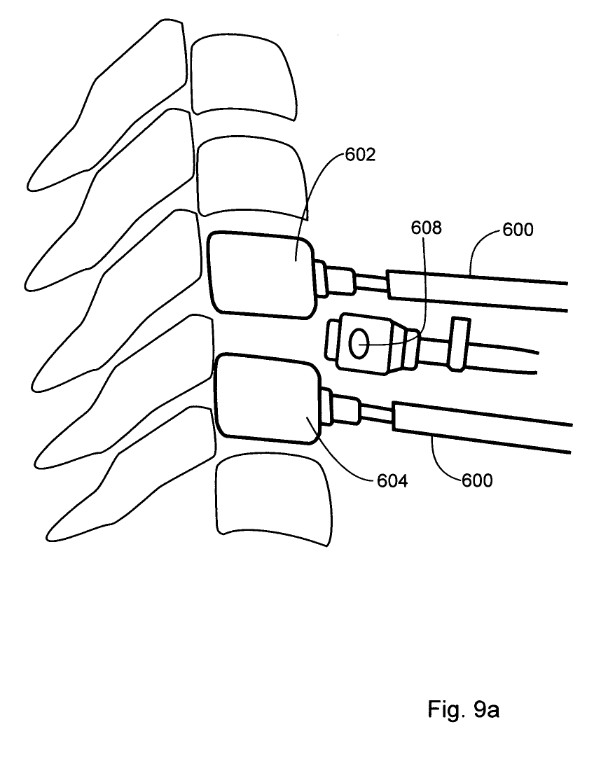

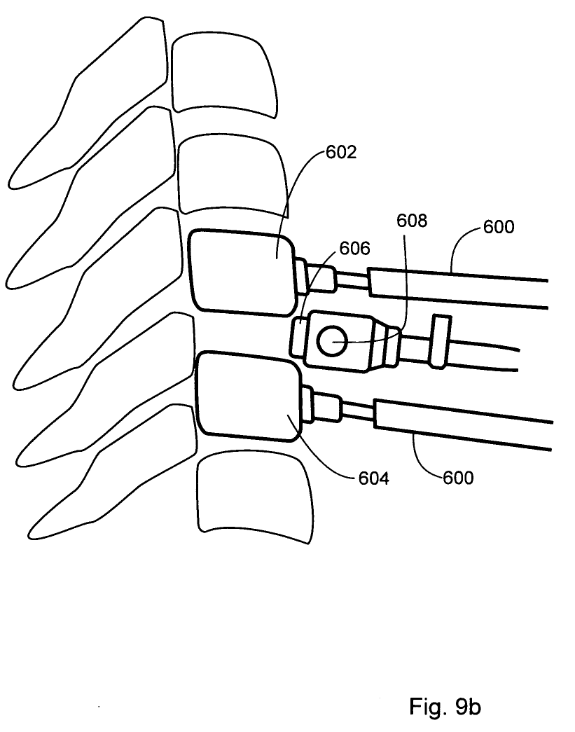

FIGS. 9A and 9B show fluoroscopic views of a variation or our chisel approaching a spine. In these views, the step of trial placement and positional adjustment of the fluoroscope have been completed. The trial has been removed. In the view shown in FIG. 9A, stabilizers have been screwed into the vertebrae (602, 604) for maintaining the spacing between the two vertebrae (602, 604). The chisel head (606) with its included side-to-side alignment port (608) may be seen. But of particular interest, the viewed shape of the alignment port (608) is not round. This indicates that the chisel head (606) is not properly aligned and is approaching the intervertebral space in an oblique direction. In FIG. 9B, the alignment port (608) is seen to be round. If the surgeon has aligned the center of the chisel head (606) with the center of the vertebrae, the fact that the alignment port (608) is seen to be round, indicates that the chisel is aligned with those vertebrae in the same way as was the trial previously aligned.

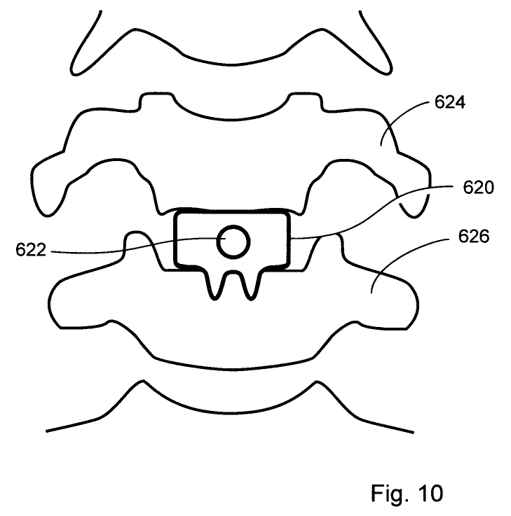

FIG. 10 shows a fluoroscopic, anterior view of our trial (620), with its handle removed, situated between an upper vertebra (624) and a lower vertebra (626). The anterior-posterior alignment opening (622) may be seen.

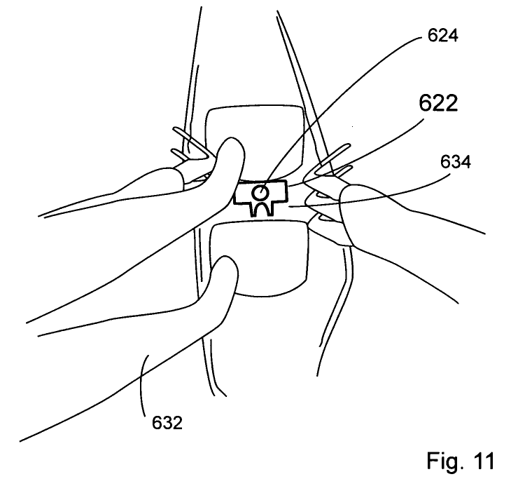

Similarly, FIG. 11 shows a fluoroscopic, anterior view of our trial (620), with its handle removed, situated between an upper vertebra (624) and a lower vertebra (626). Other surgical implements. e.g., a rib spreader with tines (630) and a stabilizer (632) affixed to the two adjacent vertebrae (624, 626) may be seen. The anterior-posterior alignment opening (622) may also be seen.

The invention is defined by the claims that follow, whether those claims are original or amended. Equivalents to those claimed inventions, as the term is defined by the courts, are considered to be within the coverage of those claims.

We claim as our invention:

1. A kit of tools for placing a prosthetic intervertebral disc in an intervertebral space between adjacent vertebrae in a human spine comprising:
a.) at least one spinal trial tool for determining an appropriate size of a prosthetic intervertebral disc for placement in the intervertebral space between adjacent vertebrae in a spine, the trial tool being alignable in a spine under fluoroscopy and comprising a trial tool head having: i.) opposing faces for engaging vertebral surfaces in the intervertebral space, ii.) opposing side surfaces joining the opposing faces, and iii.) having at least one trial alignment port extending between the opposing side surfaces, and
b.) at least one chisel tool for cutting at least one groove in at least one of the vertebral surfaces in the intervertebral space comprising a chisel tool head having: i.) opposing faces for engaging vertebral surfaces in the intervertebral space, at least one of the trial tool head opposing faces further comprising at least one cutting surface for cutting a groove in at least one of the vertebral surfaces in the intervertebral space, ii.) opposing side surfaces joining the opposing faces, and iii.) having at least one chisel alignment port extending between the opposing side surfaces.

2. The kit of claim 1 wherein the at least one chisel tool is alignable in the spine under fluoroscopy with a position of the at least one spinal trial tool defined by the at least one spinal trial tool using fluoroscopy.

3. The kit of claim 1 wherein the at least one chisel alignment port extending between the opposing side surfaces of the at least one chisel tool head is positioned substantially similar to the at least one trial alignment port extending between the opposing side surfaces of the at least one chisel tool head.

4. The kit of claim 1 wherein the trial tool head further comprises a proximal handle end and a distal end opposite the proximal handle end and an axis extending from the proximal handle end to the distal end.

5. The kit of claim 1 wherein the chisel tool head further comprises a proximal handle end and a distal end opposite the proximal handle end and an axis extending from the proximal handle end to the distal end.

6. The kit of claim 4 further comprising a handle joinable to the trial tool head at the proximal handle end.

7. The kit of claim 6 wherein the handle is removable.

8. The kit of claim 5 further comprising a handle joinable to the chisel tool head at the proximal handle end.

9. The kit of claim 8 wherein the handle is removable.

10. The kit of claim 4 wherein the at least one trial alignment port extending between the opposing side surfaces is substantially perpendicular to the axis.

11. The kit of claim 5 wherein the at least one chisel alignment port extending between the opposing side surfaces is substantially perpendicular to the axis.

12. The kit of claim 1 where the at least one trial alignment port has a cross-section selected from the group consisting of a circle, cross, rectangle, and combinations thereof.

13. The kit of claim 1 where the at least one chisel alignment port has a cross-section selected from the group consisting of a circle, cross, rectangle, and combinations thereof.

14. The kit of claim 1 wherein the trial tool head further comprises at least one front-to-back alignment port extending from the handle end to the distal end of the tool head.

15. The kit of claim 1 wherein the trial tool head opposing faces for engaging vertebral surfaces in an intervertebral space are substantially parallel.

16. The kit of claim 1 wherein the chisel tool head opposing faces for engaging vertebral surfaces in an intervertebral space are substantially parallel.

17. The kit of claim 1 wherein the trial tool head opposing faces for engaging vertebral surfaces in an intervertebral space are angled with respect to each other and taper towards each from handle end to distal end.

18. The kit of claim 1 wherein the chisel tool head opposing faces for engaging vertebral surfaces in an intervertebral space are angled with respect to each other and taper towards each from handle end to distal end.

19. The kit of claim 1 wherein at least one of the chisel tool head opposing faces further comprises at least one opening for extending a cutting surface for cutting a groove in the vertebral surface in the intervertebral space through that at least one opening and the extendable cutting surface.

20. The kit of claim 19 wherein each of the chisel tool head opposing faces further comprises at least one opening for extending a cutting surface for cutting a groove in the vertebral surface in the intervertebral space through that at least one opening and the extendable cutting surface.

21. A spinal tool comprising:
a tool head having opposing faces for engaging vertebral surfaces in an intervertebral space, wherein at least one of the opposing faces further comprises at least one opening for extending a cutting surface for cutting a groove in the vertebral surface in the intervertebral space, the at least one extendable cutting surface, a proximal handle end, a distal end opposite the proximal handle end, opposing side surfaces joining the opposing faces, and an axis extending from the proximal handle end to the distal end, and
a handle joinable to the tool head at proximal handle end.

22. The tool of claim 21 further comprising a cam movable to extend the at least one extendable cutting surface.

23. The tool of claim 21 further comprising a approachable cones movable to extend the at least one extendable cutting surface.

24. The tool of claim 21 wherein the handle is fixed to the tool head.

25. The tool of claim 21 further including a slide hammer configured to remove the tool head with extended cutting surfaces from an intervertebral opening while cutting grooves in vertebral surfaces.
